(19) United States
(12) Patent Application Publication
(10) Pub. No.: US 2011/0183961 A1
(43) Pub. Date: Jul. 28, 2011

(54) AZETIDINE POLYSUBSTITUTED COMPOUNDS, PREPARATION THEREOF, AND THERAPEUTIC APPLICATION THEREOF

(75) Inventors: Florian Auger, Paris (FR); Patrick Bernardelli, Paris (FR); Jean-François Sabuco, Paris (FR); Corinne Terrier, Paris (FR)

(73) Assignee: SANOFI-AVENTIS, Paris (FR)

(21) Appl. No.: 13/058,885

(22) PCT Filed: Aug. 13, 2009
(86) PCT No.: PCT/FR2009/001004

§ 371 Date: Apr. 11, 2011

(30) Foreign Application Priority Data
Aug. 14, 2008 (FR) 0804596

(51) Int. Cl. A61K 31/397; C07D 403/12; A61K 31/4025; C07D 409/12; C07D 401/12; A61K 31/4523; A61K 31/4178; A61P 25/04; A61P 31/12; A61P 19/00; A61P 11/00; A61P 35/00; A61P 9/00; A61P 3/00

(52) U.S. Cl. 514/210.19; 548/518; 514/422; 548/953; 546/208; 514/326; 548/314.7; 514/392

(57) ABSTRACT

The invention relates to compounds of the formula (I) where: R is a (C₁-C₆)alkyl group, a halo(C₁-C₆)alkyl group; R1 is a hydrogen atom; R2 is a heterocyclic group bound by a carbon atom, a heterocyclic-(C₁-C₄)alkyl group, said groups being optionally substituted; R3 and R4 represent independently from each other an optionally substituted phenyl group; X is a hydrogen atom, a halogen, a cyano, a (C₁-C₆)alkyl group, a halo(C₁-C₆)alkyl group, a (C₁-C₆)alkoxy group, a halo(C₁-C₆)alkoxy group or a (C₁-C₆)alkylS(0)ₚ group; and p is 0 to 2. The invention also relates to a method for preparing same and to the therapeutic application thereof.

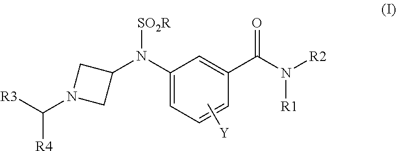

The present invention relates to azetidine derivatives, to the preparation thereof and to the therapeutic use thereof in the treatment or prevention of diseases involving CB1 cannabinoid receptors.

The subject of the present invention is compounds corresponding to formula (I)

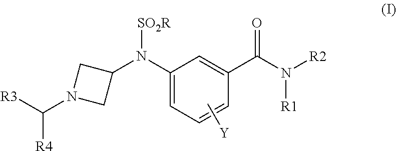

in which:
R represents a (C₁-C₆)alkyl group or a halo(C₁-C₆)alkyl group;
R1 represents hydrogen atom;
R2 represents a heterocycle group linked via a carbon atom, or a heterocycle-(C₁-C₄)alkyl group, these groups being optionally substituted with one or more atoms or groups chosen from a halogen, a hydroxyl, oxo, cyano, NH₂, C(O)NH₂, a (C₁-C₆)alkyl group, a halo(C₁-C₆)alkyl group, a (C₁-C₆)alkoxy group, a halo(C₁-C₆)alkoxy group or a COO(C₁-C₆)alkyl group;
R3 and R4 represent, independently of one another, a phenyl group optionally substituted with one or more atoms or groups chosen from a halogen, a cyano, a (C₁-C₆)alkyl group, a halo(C₁-C₆)alkyl group, a (C₁-C₆)alkoxy group or a halo(C₁-C₆)alkoxy group;
Y represents a hydrogen atom, a halogen, a cyano, a (C₁-C₆)alkyl group, a halo(C₁-C₆)alkyl group, a (C₁-C₆)alkoxy group, a halo(C₁-C₆)alkoxy group or a (C₁-C₆)alkylS(O)ₚ group;
p is between 0 and 2;
in the form of a base or of an addition salt with an acid.

The compounds of formula (I) may comprise one or more asymmetric carbon atoms. They may therefore exist in the form of enantiomers or of diastereoisomers. These enantiomers and diastereoisomers, and also mixtures thereof, including racemic mixtures, are part of the invention.

Among the compounds of formula (I) which are subjects of the invention, a first group of compounds is constituted of the compounds, as a mixture of diastereoisomers and of enantiomers, for which:

R represents a methyl,
R3 and R4 each represent a phenyl group substituted with a chlorine atom in the para-position;
Y represents hydrogen atom or a halogen;
R1 represents a hydrogen atom;
R2 represents a heterocycle group linked via a carbon atom or a heterocycle-(C₁-C₄)alkyl group and the heterocycle represents a tetrahydrothiophene, piperidine, tetrahydrothiopyran, azetidine, pyrrolidine or imidazolidine which are optionally substituted with one or more (C₁-C₆)alkyl, COO(C₁-C₆)alkyl or oxo groups; in the form of a base or of an addition salt with an acid.

The combinations of the abovementioned groups are also groups of compounds which are subjects of the invention.

In the context of the present invention:

a halogen is intended to mean a fluorine, a chlorine, a bromine or an iodine;

a (C₁-C₆)alkyl group is intended to mean a cyclic, branched or linear, saturated aliphatic group containing from 1 to 6 carbon atoms which may optionally be substituted with one or more linear, branched or cyclic (C₁-C₆)alkyl groups. By way of examples, mention may be made of methyl, ethyl, propyl, isopropyl, butyl, isobutyl, tert-butyl, pentyl, hexyl, cyclopropyl, cyclobutyl, cyclopentyl, cyclohexyl, cycloheptyl, cyclopropylmethyl groups, etc.;

a halo(C₁-C₆)alkyl group is intended to mean a (C₁-C₆)alkyl group in which one or more hydrogen atoms have been substituted with a halogen atom. By way of examples, mention may be made of CF₃, CH₂CF₃, CHF₂ and CCl₃ groups;

a hydroxy(C₁-C₆)alkyl group is intended to mean a (C₁-C₆)alkyl group in which one or more hydrogen atoms have been substituted with one or more hydroxyls;

a (C₁-C₆)alkoxy groups is intended to mean a (C₁-C₆)alkyl-O-group where the (C₁-C₆)alkyl group is as defined above:

a halo(C₁-C₆)alkoxy group is intended to mean a halo(C₁-C₆)alkyl-O-group where the halo(C₁-C₆)alkyl group is as defined above;

a heterocycle group is intended to mean a saturated or partially saturated monocyclic group containing from 4 to 6 atoms, including 1 to 3 heteroatoms chosen from O, N and S, in the knowledge that when an oxygen is present, there is at least one other heteroatom chosen from N and S. The N or S heteroatoms may be present in the oxidized form, i.e. N—O or S(O) or SO₂. By way of examples, mention may be made of piperidine, pyrrolidine, tetrahydrothiophene, imidazolidine, tetrahydrothiopyran or azetidine groups;

a heterocycle-(C₁-C₄)alkyl group is intended to mean an alkyl group substituted with a heterocycle as defined above.

The compounds of formula (I) may exist in the form of bases or of salts. Such addition salts are part of the invention.

These salts can be prepared with pharmaceutically acceptable acids, but the salts of other acids that are useful, for example, for purifying or isolating the compounds of the formula (I) are also part of the invention.

The compounds of formula (I) may also exist in the form of hydrates or of solvates, i.e. in the form of associations or combinations with one or more water molecules or with a solvent. Such hydrates and solvates are also part of the invention.

The compounds of formula (I) may also exist in the form of tautomers and are also part of the invention.

Among the compounds of formula (I) which are subjects of the invention, mention may in particular be made of the following compounds; the nomenclature used corresponds to the IUPAC nomenclature.
3-[{1-[bis(4-chlorophenyl)methyl]azetidin-3-yl}(methylsulphonyl)amino]-N-[3-(2-oxopyrrolidin-1-yl)propyl]benzamide
3-[{1-[bis(4-chlorophenyl)methyl]azetidin-3-yl}(methylsulphonyl)amino]-N-(1,1-dioxidotetrahydrothiophen-3-yl)benzamide
3-[{1-[bis(4-chlorophenyl)methyl]azetidin-3-yl}(methylsulphonyl)amino]-N-[(1-ethylpyrrolidin-2-yl)methyl]benzamide hydrochloride (1:2)
tert-butyl 4-[({3-[{1-[bis(4-chlorophenyl)methyl]azetidin-3-yl}(methyl-sulphonyl)amino]phenyl}carbonyl)amino]piperidine-1-carboxylate
(−)-3-[{1-[bis(4-chlorophenyl)methyl]azetidin-3-yl}(methylsulphonyl)amino]-N-{[1-ethyl-pyrrolidin-2-yl]methyl}benzamide
(+)-3-[{1-[bis(4-chlorophenyl)methyl]azetidin-3-yl}(methylsulphonyl)amino]-N-{[1-ethyl-pyrrolidin-2-yl]methyl}benzamide
(−)-3-[{1-[bis(4-chlorophenyl)methyl]azetidin-3-yl}(methylsulphonyl)amino]-N-[1,1-dioxidotetrahydrothiophen-3-yl]benzamide
(+)-3-[{1-[bis(4-chlorophenyl)methyl]azetidin-3-yl}(methylsulphonyl)amino]-N-[1,1-dioxidotetrahydrothiophen-3-yl]benzamide
3-[{1-[bis(4-chlorophenyl)methyl]azetidin-3-yl}(methylsulphonyl)amino]-N-[2-(2-oxoimidazolidin-1-yl)ethyl]benzamide
3-[{1-[bis(4-chlorophenyl)methyl]azetidin-3-yl}(methylsulphonyl)amino]-N-(tetrahydro-2H-thiopyran-4-yl)benzamide
3-[{1-[bis(4-chlorophenyl)methyl]azetidin-3-yl}(methylsulphonyl)amino]-N-(1,1-dioxidotetrahydro-2H-thiopyran-4-yl)benzamide
tert-butyl 3-[({3-[{1-[bis(4-chlorophenyl)methyl]azetidin-3-yl}(methyl-sulphonyl)amino]phenyl}carbonyl)amino]azetidine-1-carboxylate
3-[{1-[bis(4-chlorophenyl)methyl]azetidin-3-yl}(methylsulphonyl)amino]-N-(2-oxopyrrolidin-3-yl)benzamide
3-[{1-[bis(4-chlorophenyl)methyl]azetidin-3-yl}(methylsulphonyl)amino]-5-fluoro-N-(2-oxo-pyrrolidin-3-yl)benzamide
(+)-3-[{1-[bis(4-chlorophenyl)methyl]azetidin-3-yl}(methylsulphonyl)amino]-5-fluoro-N-[2-oxopyrrolidin-3-yl]benzamide
(−)-3-[{1-[bis(4-chlorophenyl)methyl]azetidin-3-yl}(methylsulphonyl)amino]-5-fluoro-N-[2-oxopyrrolidin-3-yl]benzamide
   the optical isomers thereof and the pharmaceutically acceptable salts thereof.

A subject of the present invention is also the use of the compounds of the invention of formula (I), for the preparation of a medicament for the treatment or prevention of diseases in which the CB1 receptor is involved.

A subject of the present invention is also the use of the compounds of the invention of formula (I), for the preparation of a medicament for the treatment or prevention of psychiatric disorders, substance dependence and withdrawal, tobacco withdrawal, cognitive and attention disorders and acute and chronic neurodegenerative diseases; metabolic disorders, appetency disorders, appetite disorders, obesity, diabetes (type I and/or II), metabolic syndrome, dyslipidaemia, sleep apnea; pain, neuropathic pain, neuropathic pain induced by anticancer drugs; gastrointestinal disorders, vomiting, ulcers, diarrhea disorders, bladder and urinary disorders, disorders of endocrine origin, cardiovascular disorders, hypotension, haemorrhagic shock, septic shock, liver diseases, chronic liver cirrhosis, fibrosis, non-alcoholic steatohepatitis (NASH), steatohepatitis and hepatic steatosis, whatever the aetiology of these conditions (alcohol, medicament, chemical product, autoimmune disease, obesity, diabetes, congenital metabolic disease); immune system diseases, rheumatoid arthritis, demyelination, multiple sclerosis, inflammatory diseases; Alzheimer's disease, Parkinson's disease, schizophrenia, cognitive disorders associated with schizophrenia, with diabetes, with obesity, with metabolic syndrome; asthma, chronic obstructive pulmonary disease, Raynaud's disease, glaucoma, fertility disorders; infectious and viral diseases, such as encephalitis, cerebral strokes, Guillain-Barré syndrome, osteoporosis and sleep apnea, and for anticancer chemotherapy; disorders related to antipsychotic treatments (weight gain, metabolic disorder).

In accordance with the invention, the compounds of general formula (I) can be prepared according to the process described in scheme 1:

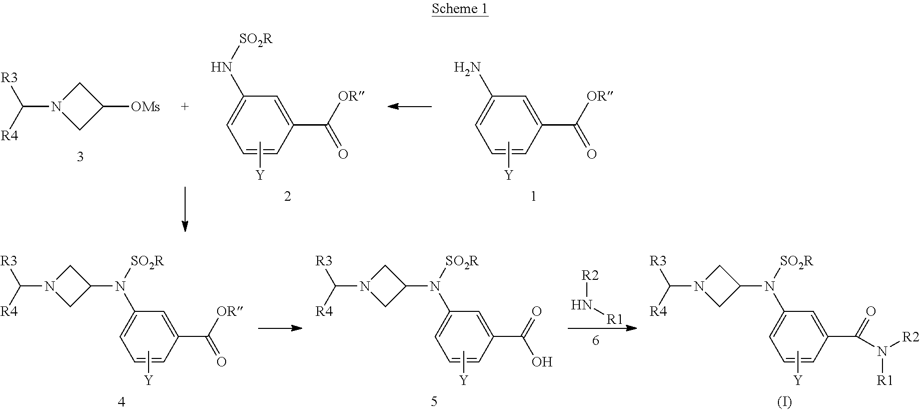

The mesylation of the compound 1 to give the derivative 2 can be carried out according to the methods known to those skilled in the art or else described in T. W. Greene, Protective Group in Organic Synthesis, fourth edition. This reaction will be carried out in a chlorinated solvent, such as dichloromethane, in the presence of a base such as pyridine and of a mesylate derivative such as mesyl chloride, at a temperature of between −10° C. and 40° C.

The derivatives 1 are commercially available or synthesized, according to the methods known to those skilled in the art, from the appropriate commercial precursors; R″ represents a protective group for the OH function of the acid.

The derivative 4 is accessible by reaction of the mesylate 2 with azetidine 3. This stage is preferably carried out under an inert atmosphere, in an inert solvent such as 4-methyl-2-pentanone, in the presence of an inorganic base such as potassium carbonate, at the reflux of the reaction mixture.

The synthesis of the azetidine 3 is described in patent application WO 01/064634.

The hydrolysis of the ester 4 to give the acid 5 is carried out according to the methods known to those skilled in the art, and more specifically in a mixture of polar solvents, such as tetrahydrofuran and water, in the presence of a base such as lithium hydroxide hydrate, at a temperature in the region of 20° C.

The compounds of formula (I) can be formed by reaction between the acid 5 and an amine derivative 6:
in a polar solvent such as tetrahydrofuran or a chlorinated solvent such as dichloromethane, in the presence or absence of a base, such as a trialkylamine (triethylamine), in the presence or absence of a coupling agent, such as 1-(3-dimethylaminopropyl)-3-ethylcarbodiimide hydrochloride or a supported carbodiimide, in the presence or absence of an additive (for example, 1-hydroxybenzotriazole),
in a polar solvent such as tetrahydrofuran or a chlorinated solvent such as dichloromethane, in the presence of a base, such as a trialkylamine (for example triethylamine or diisopropylethylamine), in the presence of an agent which promotes peptide synthesis via the formation of a mixed anhydride, such as isobutyl chloroformate,

and at a temperature of between −50° C. and the boiling point of the solvent.

The derivatives 6 are commercially available or synthesized, according to the methods known to those skilled in art, from appropriate commercial precursors. The compounds of formula (I) can be prepared by reacting an acid derivative 5 with an amine derivative 6, the reaction taking place in an inert solvent; in the presence of a coupling agent and, optionally, of an additive which prevents any racemization, optionally deprotecting the product, and then isolating the product and optionally converting it to an addition salt with an acid.

The compounds of formula (I) can be purified by the usual known methods, for example by crystallization, chromatography or extraction.

The enantiomers of the compounds of formula (I) can be obtained by resolution of the racemates, for example by chromatography on a chiral column according to Pirkle W. H. et al., Asymmetric Synthesis, vol. 1, Academic Press (1983), or by formation of salts or by synthesis from chiral precursors. The diastereoisomers can be prepared according to known conventional methods (crystallization, chromatography or from chiral precursors).

The present invention also relates to the process for preparing the intermediates.

The following examples describe the preparation of some compounds in accordance with the invention. These examples are not limiting and merely serve to illustrate the present invention. The numbers of the compounds in the examples refer to those given in the table hereinafter, which illustrates the chemical structures and the physical properties of some compounds according to the invention.

### EXAMPLE 1

### 3-[{1-[bis(4-Chlorophenyl)methyl]azetidin-3-yl}(methyl-sulphonyl)amino]-N-[3-(2-oxopyrrolidin-1-yl)propyl]benzamide (Compound No. 1)

0.5 g of 3-[{1-[bis(4-chlorophenyl)methyl]azetidin-3-yl}(methylsulphonyl)amino]benzoic acid, 10 cm³ of dichloromethane and 0.115 cm³ of 1-(3-aminopropyl)pyrrolidin-2-one are stirred at a temperature in the region of 20° C. 1.4 g of scavenger resin (PS-carbodiimide, Argonaut loading 1.3 mmol/g) are added and the reaction medium is then stirred for 20 hours at a temperature in the region of 20° C. The resin is filtered off and the filtrate is concentrated to dryness on a rotary evaporator under reduced pressure (20 kPa). The crude product obtained is purified by flash chromatography on a cartridge comprising 30 g of Merck silica (particle size: 15-40 μm; elution gradient: ethyl acetate/methanol 100/0 to 95/5). After concentration of the fractions under reduced pressure, 0.082 g of 3-[{1-[bis(4-chlorophenyl)methyl]azetidin-3-yl}(methylsulphonyl)amino]-N-[3-(2-oxopyrrolidin-1-yl)propyl]benzamide is obtained in the form of a white foam.

¹H NMR spectrum (400 MHz; (□ in ppm); (DMSO-d6); referenced at 2.50 ppm): 1.71 (m, 2H); 1.91 (m, 2H); 2.21 (t, J=8.0 Hz, 2H); 2.70 (t, J=7.5 Hz, 2H); 2.96 (s, 3H); 3.17-3.38 (partially masked m, 8H); 4.38 (s, 1H); 4.72 (m, 1H); 7.30 (d, J=9.0 Hz, 4H); 7.35 (d, J=9.0 Hz, 4H); 7.43-7.54 (m, 2H); 7.75 (broad s, 1H); 7.81 (broad d, J=8.0 Hz, 1H); 8.50 (t, J=6.5 Hz, 1H).

Mass spectrum: ES m/z=629 ([MH⁺] base peak)

Elemental analysis:

Calculated: C, 59.14%—H, 5.44%—N, 8.80%—S: 5.09%

Measured: C, 58.61%—H, 5.43%—N, 8.76%—S: 5.10%—H₂O: 1.17%

### EXAMPLE 2

### 3-[{1-[bis(4-Chlorophenyl)methyl]azetidin-3-yl}(methylsulphonyl)amino]-N-(1,1-dioxidotetrahydrothiophen-3-yl)benzamide (Compound No. 2)

0.209 g of 1-(3-dimethylaminopropyl)-3-ethylcarbodiimide hydrochloride, 0.153 cm³ of triethylamine and then 0.187 g of tetrahydrothiophen-3-amine-1,1-dioxide hydrochloride are added to a solution of 0.5 g of 3-[{1[bis(4-chlorophenyl)methyl]azetidin-3-yl}(methylsulphonyl)amino]benzoic acid in 10 cm³ of dichloromethane. The reaction medium is stirred for 24 hours under an inert atmosphere at a temperature in the region of 20° C. 20 cm³ of a saturated aqueous solution of sodium chloride are added to the reaction medium. After separation by settling out, the aqueous phase is extracted with dichloromethane. The organic phases are combined, dried over magnesium sulphate, filtered, and concentrated to dryness on a rotary evaporation under reduced pressure (5 kPa). 0.587 g of product is obtained, and is purified by flash chromatography on a cartridge comprising 30 g of Merck silica (particle size: 15-40 μm; eluent: 100 ethyl acetate). After concentration of the fractions under reduced pressure, 0.246 g of 3-[{1-[bis(4-chlorophenyl)methyl]azetidin-3-yl}(methylsulphonyl)amino]-N-(1,1-dioxidotetrahydrothiophen-3-yl)benzamide is obtained in the form of a white foam.

¹H NMR spectrum (300 MHz; (□ in ppm); (DMSO-d6); referenced at 2.50 ppm): 2.21 (m, 1H); 2.43 (partially masked m, 1H); 2.69 (t, J=7.5 Hz, 2H); 2.97 (s, 3H); 3.08 (dd, J=8.0; 13.0 Hz, 1H); 3.12-3.43 (partially masked m, 4H); 3.50 (dd, J=8.0; 13.0 Hz, 1H); 4.37 (s, 1H); 4.60-4.80 (m, 2H); 7.30 (d, J=9.0 Hz, 4H); 7.36 (d, J=9.0 Hz, 4H); 7.45-7.57 (m, 2H); 7.78 (s, 1H); 7.83 (m, 1H); 8.78 (d, J=7.0 Hz, 1H)

Mass spectrum: ES m/z=622 ([MH⁺], base peak)

Elemental analysis:

Calculated: C, 54.02%—H, 4.70%—N, 6.75%—S: 10.30%—Cl: 11.39%

Measured: C, 53.50%—H, 4.27%—N, 6.63%—S: 10.44%—Cl: 11.71%—H₂O: 1.28%

### EXAMPLE 3

### (−)-3-[{1-[bis(4-Chlorophenyl)methyl]azetidin-3-yl}(methyl-sulphonyl)amino]-N-(1,1-dioxidotetrahydrothiophen-3-yl)benzamide (Compound No. 7)

601 mg of 3-[{1-[bis(4-chlorophenyl)methyl]azetidin-3-yl}(methylsulphonyl)amino]-N-(1,1-dioxidotetrahydrothiophen-3-yl)benzamide are injected onto a column containing 700 g of chiral stationary phase, chirobiotic TAG 10 μm. The elution is carried out at 130 cm³ per minute with 100% methanol as eluent. The laevorotatory enantiomer is eluted first. After concentration of the solvent, 206 mg of (−)-3-[{1-[bis(4-chlorophenyl)methyl]azetidin-3-yl}(methylsulphonyl)amino]-N-[1,1-dioxidotetrahydrothiophen-3-yl]benzamide are obtained in the form of a white power.

¹H NMR spectrum (400 MHz; (□ in ppm); (DMSO-d6); referenced at 2.50 ppm): 2.21 (m, 1H); 2.43 (partially masked m, 1H); 2.70 (m, 2H); 2.96 (s, 3H); 3.08 (dd, J=13.7; 7.8 Hz, 1H); 3.17-3.54 (partially masked m, 5H); 4.37 (s, 1H); 4.03-4.79 (m, 2H); 7.31 (d, J=8.8 Hz, 4H); 7.35 (d, J=8.8 Hz, 4H); 7.46-7.54 (m, 2H); 7.77 (broad s, 1H); 7.84 (m, 1H); 8.77 (d, J=7.1 Hz, 1H)

Mass spectrum: ES m/z=622 [M+H]⁺; m/z=620 [M−H]⁻

Elemental analysis:

Calculated: C, 54.02%—H, 4.70%—N, 6.75%—S: 10.30%

Measured: C, 53.82%—H, 4.94%—N, 6.65%—S: 9.81%—H₂O: 1.00%

Optical rotation: □_{D}=−21.1+/−0.8 (c=0.346, DMSO)

### EXAMPLE 4

### (+)-3-[{1-[bis(4-Chlorophenyl)methyl]azetidin-3-yl}(methylsulphonyl)amino]-N-[1,1-dioxidotetrahydrothiophen-3-yl]benzamide (Compound No. 8)

The dextrorotatory enantiomer was eluted in second position during the separation carried out in Example 3. After concentration of the solvent, 176 mg of (+)-3-[{1-[bis(4-chlorophenyl)methyl]azetidin-3-yl}(methylsulphonyl)amino]-N-[1,1-dioxidotetrahydrothiophen-3-yl]benzamide are obtained in the form of a white power.

¹H NMR spectrum (400 MHz; (□ in ppm); (DMSO-d6); referenced at 2.50 ppm): 2.21 (m, 1H); 2.43 (partially masked m, 1H); 2.70 (m, 2H); 2.96 (s, 3H); 3.07 (dd, J=13.7; 7.8 Hz, 1H); 3.15-3.41 (partially masked m, 4H); 3.49 (dd, J=13.7; 8.1 Hz, 1H); 4.37 (s, 1H); 4.63-4.79 (m, 2H); 7.31 (d, J=8.8 Hz, 4H); 7.35 (d, J=8.8 Hz, 4H); 7.46-7.54 (m, 2H); 7.77 (broad s, 1H); 7.84 (m, 1H); 8.76 (d, J=7.3 Hz, 1H)

Mass spectrum: ES m/z=622 [M+H]⁺; m/z=620 [M−H]⁻

Elemental analysis:

Calculated: C, 54.02%—H, 4.70%—N, 6.75%—S: 10.30%

Measured: C, 53.68%—H, 4.77%—N, 6.90%—S: 9.68%—H₂O: 1.69%

Optical rotation: □_{D}=+11.5+/−0.5 (c=0.391, DMSO)

### EXAMPLE 5

### 3-[{1-[bis(4-Chlorophenyl)methyl]azetidin-3-yl}(methylsulphonyl)amino]-N-[(1-ethylpyrrolidin-2-yl)methyl]benzamide hydrochloride (1:2) (Compound No. 3)

### 5a: 3-[{1-[bis(4-Chlorophenyl)methyl]azetidin-3-yl}(methylsulphonyl)amino]-N-[(1-ethylpyrrolidin-2-yl)methyl]benzamide

0.177 cm³ of isobutyl chloroformate is added, dropwise, under an inert atmosphere, at a temperature in the region of −5° C., to a solution of 0.6 g of 3-[{1-[bis(4-chlorophenyl)methyl]azetidin-3-yl}(methylsulphonyl)amino]benzoic acid, 20 cm³ of tetrahydrofuran and 0.217 cm³ of triethylamine. The suspension obtained is stirred for 40 minutes at a temperature below 10° C. 0.255 cm³ of 1-(1-ethylpyrrolidin-2-yl)methanamine is added at a temperature in the region of −5° C. The mixture is allowed to return gradually to a temperature in the region of 20° C., and then stirred for 24 hours at this temperature. A saturated aqueous solution of sodium chloride is added to the reaction medium. After separation by settling out, the aqueous phase is extracted with dichloromethane. The organic phases are combined, dried over magnesium sulphate, filtered, and concentrated to dryness on a rotary evaporator under reduced pressure (5 kPa). 0.972 g of crude product is obtained and is purified by flash chromatography on a cartridge comprising 90 of Merck silica (particle size: 15-40 μm; eluent: dichloromethane/methanol 96/4). After concentration of the fractions under reduced pressure, 0.248 g of 3-[{1-[bis(4-chloro-phenyl)methyl]azetidin-3-yl}(methylsulphonyl)amino]-N-[(1-ethylpyrrolidin-2-yl)methyl]benzamide is obtained.

Mass spectrum: ES m/z=615 (MH⁺)

### 5b: 3-[{1-[bis(4-Chlorophenyl)methyl]azetidin-3-yl}(methylsulphonyl)amino]-N-[(1-ethylpyrrolidin-2-yl)methyl]benzamide hydrochloride (1:2) (Compound No. 3)

After filtration of the suspension of 0.195 g of 3-[{1-[bis(4-chlorophenyl)methyl]azetidin-3-yl}(methylsulphonyl)amino]-N-[(1-ethylpyrrolidin-2-yl)methyl]benzamide in dichloromethane, 1.58 cm³ of 1N solution of hydrochloric acid in ethyl ether are added. The reaction medium is concentrated to dryness on a rotary evaporator under reduced pressure (5 kPa). 0.19 g of 3-[{1-[bis(4-chlorophenyl)methyl]azetidin-3-yl}(methylsulphonyl)amino]-N-[(1-ethylpyrrolidin-2-yl)-methyl]benzamide hydrochloride is obtained in the form of a white solid.

¹H NMR spectrum (400 MHz; (□ in ppm); (DMSO-d6); referenced at 2.50 ppm): for this batch, we observe a 70%—30% mixture of conformers and salification with 2 HCl with: 129 (t, J=6.5 Hz, 3H); from 1.75 to 2.03 (m, 3H); 2.12 (m, 1H); 3.02 (s, 3H); from 3.03 to 3.15 (m, 2H); 3.43 (m, 1H); 3.54 (m, 1H); 3.63 (m, 2H); from 3.70 to 4.18 (partially masked m, 5H); 5.05 (broad m, 0.7H, 5.48 (broad m, 0.3H); 5.95 (broad m, 0.7H); 6.10 (broad m, 0.3H); from 7.30 to 7.75 (m, 10H); from 7.90 to 8.03 (m, 2H); 9.15 (t, J=6.0 Hz, 1H); 10.1 (broad m, 0.3H); 10.2 (broad m, 0.7H); 12.65 (broad m, 0.3H); 13.05 (broad m, 0.7H).

Mass spectrum: ES m/z=615 (MH⁺); m/z=381 ([MH−C₁₃H₉Cl₂+H]⁺, base peak);

m/z=235 (C₁₃H₉Cl₂⁺)

Elemental analysis:

Calculated: C, 57.10%—H, 5.72%—N, 8.59%—S: 4.92%—Cl: 16.31%

Measured: C, 52.955%—H, 5.99%—N, 7.40%—S: 4.18%—Cl: 19.90%—H₂O: 2.21%

### EXAMPLE 6

### (+)-3-[{1[bis(4-Chlorophenyl)methyl]azetidin-3-yl}(methyl-sulphonyl)amino]-N-{[1-ethylpyrrolidin-2-yl]methyl}benzamide (Compound No. 6)

84 mg of (R)-(+)-2-aminomethyl-1-ethylpyrrolidine are added to a solution of 0.3 g of 3-[{1-[bis(4-chlorophenyl)methyl]azetidin-3-yl}(methylsulphonyl)amino]benzoic acid in 3 cm³ of dichloromethane. The reaction medium is stirred for 10 minutes at a temperature in the region of 20° C., before the addition of 136 mg of 1-(3-dimethylaminopropyl)-3-ethylcarbodiimide hydrochloride. After stirring overnight at a temperature in the region of 20° C., the reaction medium is diluted with 25 cm³ of water and 20 cm³ of dichloromethane. After separation by settling out, the aqueous phase is extracted twice with 20 cm³ of dichloromethane. The combined organic phases are dried, filtered, and then concentrated to dryness under reduced pressure. The reaction crude obtained is purified by flash chromatography on a column comprising 30 g of silica (elution gradient: acetonitrile/methanol: up to 80/20). After concentration of the fractions under reduced pressure, a white foam is obtained, which is solubilized in a minimum of dichloromethane. Heptane is added to this solution until it becomes cloudy. After concentration of the suspension under vacuum and drying in an oven overnight, 137 mg of (+)-3-[{1-[bis(4-chlorophenyl)methyl]azetidin-3-yl}(methylsulphonyl)amino]-N-{[1-ethylpyrrolidin-2-yl]methyl}benzamide are obtained in the form of white foam.

Mp: 122° C.

¹H NMR spectrum (400 MHz; (0 in ppm); (DMSO-d6); referenced at 2.50 ppm): 1.02 (t, J=7.1 Hz, 3H); 1.51-1.67 (m, 3H); 1.77 (m, 1H); 2.12 (m, 1H); 2.26 (m, 1H); 2.58 (m, 1H); 2.70 (t, J=6.7 Hz, 2H); 2.81 (m, 1H); 2.96 (s, 3H); 3.01 (m, 1H); 3.07 (m, 1H); 3.24-3.42 (partially masked m, 3H); 4.37 (s, 1H); 4.73 (m, 1H); 7.30 (d, J=8.8 Hz, 4H); 7.35 (d, J=8.8 Hz, 4H); 7.43-7.53 (m, 2H); 7.75 (t, J=1.8 Hz, 1H); 7.80 (dt, J=7.5; 1.8 Hz, 1H); 8.45 (t, J=5.9 Hz, 1H)

Mass spectrum: ES m/z=615 [M+H]⁺; m/z=381 ([M−C₁₃H₈Cl₂+H]⁺, base peak);

m/z=613 [M−H]⁻; m/z=659 ([M+HCO₂H−H]⁻, base peak)

Optical rotation: □_{D}=+24.5+/−0.8 (c=0.349, MeOH)

### EXAMPLE 7

### (−)-3-[{1-[bis(4-Chlorophenyl)methyl]azetidin-3-yl}(methyl-sulphonyl)amino]-N-{[1-ethylpyrrolidin-2-yl]methyl}benzamide (Compound No. 5)

(−)-3-[{1-[bis(4-Chlorophenyl)methyl]azetidin-3-yl}(methylsulphonyl)amino]-N-{[1-ethylpyrrolidin-2-yl]methyl}benzamide is synthesized as described in Example 6, starting from 0.3 g of 3-[{1-[bis(4-chlorophenyl)methyl]azetidin-3-yl}(methylsulphonyl)amino]benzoic acid, 3 cm³ of dichloromethane, 84 mg of (S)-(−)-2-aminomethyl-1-ethylpyrrolidine and 136 mg of 1-(3-dimethylaminopropyl)-3-ethylcarbodiimide hydrochloride. After reaction, treatment and purification, 153 mg of (−)-3-[{1-[bis(4-chlorophenyl)methyl]azetidin-3-yl}(methylsulphonyl)amino]-N-{[1-ethyl-pyrrolidin-2-yl]methyl}benzamide are obtained in the form of a white foam.

Mp: 128° C.

¹H NMR spectrum (400 MHz; (E in ppm); (DMSO-d6); referenced at 2.50 ppm): 1.02 (t, J=7.1 Hz, 3H); 1.51-1.67 (m, 3H); 1.77 (m, 1H); 2.12 (m, 1H); 2.27 (m, 1H); 2.58 (m, 1H): 2.70 (t, J=6.7 Hz, 2H); 2.81 (m, 1H); 2.96 (s, 3H); 3.01 (m, 1H); 3.07 (m, 1H); 3.24-3.42 (partially masked m, 3H); 4.37 (s, 1H); 4.73 (m, 1H); 730 (d, J=8.8 Hz, 4H); 7.35 (d, J=8.8 Hz, 4H); 7.43-7.53 (m, 2H); 7.75 (t, J=1.8 Hz, 1H); 7.80 (dt, J=7.5; 1.8 Hz, 1H); 8.45 (t, J=5.9 Hz, 1H)

Mass spectrum ES m/z=615 [M+H]⁺, m/z=381 ([M−C₁₃H₈Cl₂+H]⁺, base peak),

m/z=613 [M−H]⁻, m/z=659 ([M+HCO₂H−H]⁻, base peak)

Optical rotation: □_{D}=−22+/−0.9 (c=0.284, MeOH)

### EXAMPLE 8

### 3-[{1[bis(4-Chlorophenyl)methyl]azetidin-3-yl}(methyl-sulphonyl)amino]-5-fluoro-N-(2-oxopyrrolidin-3-yl)benzamide (Compound No. 14)

0.333 cm³ of triethylamine and 0.135 cm³ of isobutyl chloroformate are successively added to a solution of 0.50 g of 3-[{1-[bis(4-chlorophenyl)methyl]azetidin-3-yl}(methylsulphonyl)amino)-5-fluorobenzoic acid in 10 cm³ of tetrahydrofuran, stirred at a temperature in the region of −30° C. The reaction medium is stirred for 1 hour while bringing the temperature back from −30° C. to 0° C., and then for 30 minutes while bringing the temperature back from 0° C. to 4° C. 144 mg of 3-amino-2-pyrrolidone and 5 cm³ of tetrahydrofuran are then added. After stirring for 19 hours at a temperature in the region of 20° C., the reaction medium is cooled to a temperature in the region of −20° C., before hydrolysis with 15 cm³ of water. The medium is subsequently stirred for 1 hour at a temperature in the region of 20° C. and then extracted 3 times with 20 cm³ of ethyl acetate. The organic phases are combined, dried over magnesium sulphate, and then filtered before concentration to dryness. 590 mg of a yellow foam are obtained, and said foam is purified by flash chromatography on a column comprising 30 g of silica (Merck, 15-40 μm, eluent: ethyl acetate/methanol 98/2). After concentration of the fractions under reduced pressure, 282 mg of 3-[{1-[bis(4-chlorophenyl)methyl]azetidin-3-yl}(methylsulphonyl)amino]-5-fluoro-N-(2-oxopyrrolidin-3-yl)benzamide are obtained in the form of a white foam.

¹H NMR spectrum (400 MHz; (□ in ppm); (DMSO-d6); referenced at 2.50 ppm): 2.01 (m, 1H); 2.36 (m, 1H); 2.74 (m, 2H); 3.00 (s, 3H); 3.24 (m, 2H); 3.36 (m, 2H); 4.40 (s, 1H); 4.54 (m, 1H); 4.72 (m, 1H); 7.30-7.38 (m, 8H); 7.44 (dt, J=9.3; 2.1 Hz, 1H); 7.67 (broad s, 1H); 7.68 (m, 1H); 7.86 (s, 1H); 8.83 (d, J=8.3 Hz, 1H)

Mass spectrum: ES m/z=605 [M+H]⁺; m/z=603 [M−H]⁻; m/z=649 [M+HCO₂H−H]⁻

Elemental analysis:

Calculated: C, 55.54%—H, 4.49%—N, 9.25%—S: 5.30%

Measured: C, 55.77%—H, 4.72%—N, 8.91%—S: 4.93

### EXAMPLE 9

### (+)-3-[{1-[bis(4-Chlorophenyl)methyl]azetidin-3-yl}(methylsulphonyl)amino]-5-fluoro-N-[2-oxopyrrolidin-3-yl]benzamide (Compound No. 15)

990 mg of 3-[{1-[bis(4-chlorophenyl)methyl]azetidin-3-yl}(methylsulphonyl)amino]-5-fluoro-N-(2-oxopyrrolidin-3-yl)benzamide are injected onto a column containing a chiral stationary phase, Chiralpak IA 20 μm. The elution is carried out at 120 cm³ per minute with a 90/10 mixture of acetonitrile/isopropanol as eluent. The dextrorotatory enantiomer is eluted in first position. After concentration of the solvent, 360 mg of (+)-3-[{1-[bis(4-chlorophenyl)methyl]azetidin-3-yl}(methylsulphonyl)amino]-5-fluoro-N-[2-oxopyrrolidin-3-yl]benzamide are obtained in the form of a white foam.

¹H NMR spectrum (400 MHz; (□ in ppm); (DMSO-d6); referenced at 2.50 ppm: 2.00 (m, 1H); 2.35 (m, 1H); 2.74 (m, 2H); 3.00 (s, 3H); 3.25 (m, 2H); 3.36 (m, 2H); 4.40 (s, 1H); 4.53 (m, 1H); 4.72 (m, 1H); 7.31 (d, J=8.6 Hz, 4H); 7.36 (d, J=8.6 Hz, 4H); 7.44 (broad d, J=9.5 Hz, 1H); 7.64-7.74 (m, 2H); 7.87 (broad d, 1H); 8.84 (broad d, J=8.6 Hz, 1H)

Mass spectrum: ES m/z=605 [M+H]⁺; m/z=603 [M−H]⁻

Elemental analysis:

Calculated: C, 55.54%—H, 4.49%—N, 9.25%—S: 5.30%

Measured: C, 55.32%—H, 4.86%—N, 8.92%—S: 5.06%

Optical rotation: □_{D}=+7.4+/−0.5 (c=0.482, DMSO)

### EXAMPLE 10

### (−)-3-[{1-[bis(4-Chlorophenyl)methyl]azetidin-3-yl}(methylsulphonyl)amino]-5-fluoro-N-[2-oxopyrrolidin-3-yl]benzamide (Compound No. 16)

The laevorotatory enantiomer is eluted in second position. After concentration of the solvent, 466 mg of (−)-3-[{1-[bis(4-chlorophenyl)methyl]azetidin-3-yl}(methylsulphonyl)amino]-5-fluoro-N-[2-oxopyrrolidin-3-yl]benzamide are obtained in the form of a white foam.

¹H NMR spectrum (400 MHz; (□ in ppm); (DMSO-d6); referenced at 2.50 ppm): 2.00 (m, 1H); 2.34 (m, 1H); 2.74 (m, 2H); 3.00 (s, 3H); 3.25 (m, 2H); 3.36 (m, 2H); 4.40 (s, 1H); 4.53 (m, 1H); 4.72 (m, 1H); 7.31 (d, J=8.6 Hz, 4H); 7.35 (d, J=8.6 Hz, 4H); 7.44 (broad d, J=9.3 Hz, 1H); 7.62-7.72 (m, 2H); 7.87 (broad s, 1H); 8.84 (d, J=8.3 Hz, 1H)

Mass spectrum: ES m/z=605 [M+H]⁺; m/z=603 [M+H]⁺; m/z=649 [M+HCO₂H−H]⁻

Elemental analysis:

Calculated: C, 55.54%—H, 4.49%—N, 9.25%—S: 5.30%

Measured: C, 55.21%—H, 4.73%—N, 9.07%—S: 4.95%

Optical rotation: □_{D}=−9.4+/−0.6 (c=0.433, DMSO)

Table 1 which follows illustrates the chemical structures (I) and the physical properties of some examples of compounds according to the invention. In this table:
R represents a methyl group;
R3 and R4 each represent a phenyl group substituted with a chlorine atom in the para-position;

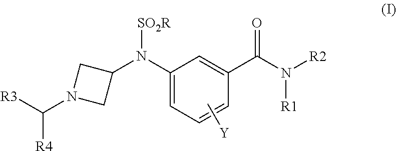

TABLE 1

| No | 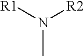 | Y | Chirality/salt/characterizations |
|---|---|---|---|
| 1 | 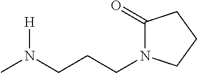 | H | ¹H NMR spectrum (400 MHz; (□ in ppm); (DMSO- d6); referenced at 2.50 ppm); 1.71 (m, 2H); 1.91 (m, 2H); 2.21 (t, J = 8.0 Hz, 2H); 2.70 (t, J = 7.5 Hz, 2H); 2.96 (s, 3H); 3.17-3.38 (partially masked m, 8H); 4.38 (s, 1H); 4.72 (m, 1H); 7.30 (d, J = 9.0 Hz, 4H); 7.35 (d, J = 9.0 Hz, 4H); 7.43-7.54 (m, 2H); 7.75 (broad s, 1H); 7.81 (broad d, J = 8.0 Hz, 1H); 8.50 (t, J = 6.5 Hz, 1H); Mass spectrum: ES: m/z = 629 (MH⁺, base peak); Elemental analysis: Calculated: C: 59.14%- H: 5.44%- N: 8.90%- S: 5.09%; Measured: C: 58.61%- H: 5.43%- N: 8.76%- S: 5.10%- H₂O: 1.17% |
| 2 | 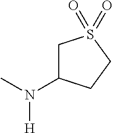 | H | ¹H NMR spectrum (300 MHz; (□ in ppm); (DMSO- d6); referenced at 2.50 ppm): 2.21 (m, 1H); 2.43 (partially masked m, 1H); 2.69 (t, J = 7.5 Hz, 2H); 2.97 (s, 3H); 3.08 (dd, J = 8.0; 13.0 Hz, 1H); 3.12- 3.43 (partially masked m, 4H); 3.50 (dd, J = 8.0; 13.0 Hz, 1H); 4.37 (s, 1H); 4.60-4.80 (m, 2H); 7.30 (d, J = 9.0 Hz, 4H); 7.36 (d, J = 9.0 Hz, 4H); 7.45-7.57 (m, 2H); 7.78 (s, 1H); 7.83 (m, 1H); 8.78 (d, J = 7.0 Hz, 1H); Mass spectrum: ES: m/z = 622 (MH⁺, base peak); Elemental analysis: Calculated: C: 54.02%- H: 4.70%- N: 6.75%- S: 10.3%- Cl 11.39%; Measured: C: 53.50%- H: 4.27%- N: 6.63%- S: 10.44%- Cl 11.71%- H₂O: 1.28% |
| 3 | 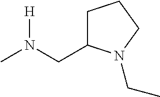 | H | 2 HCl; ¹H NMR spectrum (400 MHz; (□ in ppm); (DMSO- d6); referenced at 2.50 ppm): For this batch, we observe a 70%-30% mixture of confomers and salification with 2 HCl with: 1.29 (t, J = 6.5 Hz, 3H); 1.75-2.03 (m, 3H); 2.12 (m, 1H); 3.02 (s, 3H); 3.03-3.15 (m, 2H); 3.43 (m, 1H); 3.54 (m, 1H); 3.63 (m, 2H); 3.70-4.18 (partially masked m, 5H); 5.05 (broad m, 0.7H); 5.48 (broad m, 0.3H); 5.95 (broad m, 0.7H); 6.10 (broad m, 0.3H); 7.30-7.75 (m, 10H); 7.90-8.03 (m, 2H); 9.15 (t, J = 6.0 Hz, 1H); 10.1 (broad m, 0.3H); 10.2 (broad m, 0.7H); 12.65 (broad m, 0.3H); 13.05 (broad m, 0.7H); Mass spectrum: ES: m/z = 615 (MH⁺), m/z = 381 (MH − C₁₃H₉Cl₂ + H)⁺, base peak), m/z = 235 (C₁₃H₉Cl₂⁺) |
| 4 | 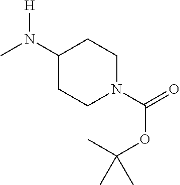 | H | ¹H NMR spectrum (400 MHz; (□ in ppm); (DMSO- d6); referenced at 2.50 ppm): 1.38 (m, 9H); 1.76 (m, 2H); 2.89-2.60 (m, 4H); 2.92 (s, 3H); 3.28 (m, 4H); 3.92 (m, 3H); 4.34 (s, 1H); 4.70 (m, 1H); 7.30 (m, 8H); 7.45 (m, 2H); 7.73 (m, 1H); 7.79 (m, 1H); 8.27 (d, J = 8 Hz, 1H); Mass spectrum: ES m/z = 686 (MH⁺) |
| 5 | 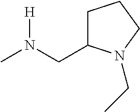 | H | Chiral (−); Mp: 128° C.; ¹H NMR spectrum (400 MHz; (□ in ppm); (DMSO-d6); referenced at 2.50 ppm): 1.02 (t, J = 7.1 Hz, 3H); 1.51-1.67 (m, 3H); 1.77 (m, 1H); 2.12 (m, 1H); 2.27 (m, 1H); 2.58 (m, 1H); 2.70 (t, J = 6.7 Hz, 2H); 2.81 (m, 1H); 2.96 (s, 3H); 3.01 (m, 1H); 3.07 (m, 1H); 3.24-3.42 (partially masked m, 3H); 4.37 (s, 1H); 4.73 (m, 1H); 7.30 (d, J = 8.8 Hz, 4H); 7.35 (d, J = 8.8 Hz, 4H); 7.43- 7.53 (m, 2H); 7.75 (t, J = 1.8 Hz, 1H); 7.80 (dt, J = 7.5; 1.8 Hz, 1H); 8.45 (t, J = 5.9 Hz, 1H); Mass spectrum: ES: m/z = 615 [M + H]⁺, m/z = 381 ([M − C₁₃H₈Cl₂ + H]⁺, base peak), m/z = 613 [M − H]⁻, m/z = 659 ([M + HCO₂H − H]⁻, base peak); Optical rotation: □_{D} = −22 +/− 0.9 (c = 0.284, MeOH) |
| 6 | 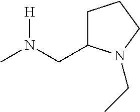 | H | Chiral (+); Mp: 122° C.; ¹H NMR spectrum (400 MHz; (□ in ppm); (DMSO-d6); referenced at 2.50 ppm): 1.02 (t, J = 7.1 Hz, 3H); 1.51-1.67 (m, 3H); 1.77 (m, 1H); 2.12 (m, 1H); 2.26 (m, 1H); 2.58 (m, 1H); 2.70 (t, J = 6.7 Hz, 2H); 2.81 (m, 1H); 2.96 (s, 3H); 3.01 (m, 1H); 3.07 (m, 1H); 3.24-3.42 (partially masked m, 3H); 4.37 (s, 1H); 4.73 (m, 1H); 7.30 (d, J = 8.8 Hz, 4H); 7.35 (d, J = 8.8 Hz, 4H); 7.43- 7.53 (m, 2H); 7.75 (t, J = 1.8 Hz, 1H); 7.80 (dt, J = 7.5; 1.8 Hz, 1H); 8.45 (t, J = 5.9 Hz, 1H); Mass spectrum: ES: m/z = 615 [M + H]⁺; m/z = 381 ([M − C₁₃H₈Cl₂ + H]⁺, base peak), m/z = 613 [M − H]⁻; m/z = 659 ([M + HCO₂H − H]⁻, base peak); Optical rotation: □_{D} = +24.5 +/− 0.8 (c = 0.349, MeOH) |
| 7 | 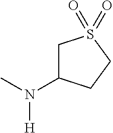 | H | Chiral (−); ¹H NMR spectrum (400 MHz; (□ in ppm); (DMSO- d6); referenced at 2.50 ppm): 2.21 (m, 1H); 2.43 (partially masked m, 1H); 2.70 (m, 2H); 2.96 (s, 3H); 3.08 (dd, J = 13.7; 7.8 Hz, 1H); 3.17-3.54 (partially masked m, 5H); 4.37 (s, 1H); 4.63-4.79 (m, 2H); 7.31 (d, J = 8.8 Hz, 4H); 7.35 (d, J = 8.8 Hz, 4H); 7.46-7.54 (m, 2H); 7.77 (broad s, 1H); 7.84 (m, 1H); 8.77 (d, J = 7.1 Hz, 1H); Mass spectrum: ES: m/z = 622 [M + H]⁺; m/z = 620 [M − H]⁻; Elemental analysis: Calculated: C: 54.02%- H: 4.70%- N: 6.75%- S: 10.3%; Measured: C: 53.82%- H: 4.94%- N: 6.65%- S: 9.81%- H₂O: 1.00%; Optical rotation: □_{D} = −21.1 +/− 0.8 (c = 0.346, DMSO) |
| 8 | 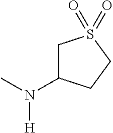 | H | Chiral (+); ¹H NMR spectrum (400 MHz; (□ in ppm); (DMSO- d6); referenced at 2.50 ppm): 2.21 (m, 1H); 2.43 (partially masked m, 1H); 2.70 (m, 2H); 2.96 (s, 3H); 3.07 (dd, J = 13.7; 7.8 Hz, 1H); 3.15-3.41 (partially masked m, 4H); 3.49 (dd, J = 13.7; 8.1 Hz, 1H); 4.37 (s, 1H); 4.63-4.79 (m, 2H); 7.31 (d, J = 8.8 Hz, 4H); 7.35 (d, J = 8.8 Hz, 4H); 7.46-7.54 (m, 2H); 7.77 (broad s, 1H); 7.84 (m, 1H); 8.76 (d, J = 7.3 Hz, 1H); Mass spectrum: ES: m/z = 622 [M + H]⁺; m/z = 620 [M − H]⁻; Elemental analysis: Calculated: C: 54.02%- H: 4.70%- N: 6.75%- S: 10.30%; Measured: C: 53.68%- H: 4.77%- N: 6.90%- S: 9.68%- H₂O: 1.69%; Optical rotation: □_{D} = +11.5 +/− 0.5 (c = 0.391, DMSO) |
| 9 | 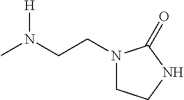 | H | Mp: 130° C.; ¹H NMR spectrum (400 MHz; (□ in ppm); (DMSO-d6); referenced at 2.50 ppm): 2.70 (t, J = 7.2 Hz, 2H); 2.96 (s, 3H); 3.16-3.25 (m, 4H); 3.28-3.42 (partially masked m, 6H); 4.37 (s, 1H); 4.72 (m, 1H); 6.26 (s, 1H); 7.31 (d, J = 8.8 Hz, 4H); 7.35 (d, J = 8.8 Hz, 4H); 7.44-7.51 (m, 2H); 7.71 (broad s, 1H); 7.78 (broad d, J = 7.7 Hz, 1H); 8.58 (t, J = 5.7 Hz, 1H); Mass spectrum: ES: m/z = 616 ([M + H]⁺; base peak); m/z = 1231 [2M + H]⁺ |
| 10 | 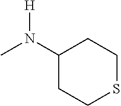 | H | Mp: 210° C.; ¹H NMR spectrum (400 MHz; (□ in ppm); (DMSO-d6); referenced at 2.50 ppm): 1.66 (m, 2H); 2.09 (m, 2H); 2.60-2.77 (m, 6H); 2.96 (s, 3H); 3.33 (partially masked m, 2H); 3.82 (m, 1H); 4.37 (s, 1H); 4.74 (m, 1H); 7.30 (d, J = 8.8 Hz, 4H); 7.35 (d, J = 8.8 Hz, 4H); 7.44-7.53 (m, 2H); 7.76 (broad s, 1H); 7.82 (m, 1H); 8.37 (d, J = 8.1 Hz, 1H); Mass spectrum: ES: m/z = 604 ([M + H]⁺, base peak); m/z = 1206 [2M + H]⁺; Elemental analysis: Calculated: C: 57.61%- H: 5.17%- N: 6.95%- S: 10.61%; Measured: C: 57.39%- H: 5.26%- N: 6.88%- S: 10.32% |
| 11 | 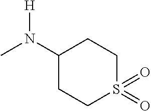 | H | Mp: 270° C.; ¹H NMR spectrum (400 MHz; (□ in ppm); (DMSO-d6); referenced at 2.50 ppm): 2.01- 2.17 (m, 4H); 2.70 (t, J = 7.3 Hz, 2H); 2.96 (s, 3H); 3.11 (m, 2H); 3.27-3.39 (m, 4H); 4.21 (m, 1H); 4.38 (s, 1H); 4.74 (m, 1H); 7.31 (d, J = 8.8 Hz, 4H); 7.35 (d, J = 8.8 Hz, 4H); 7.45-7.53 (m, 2H); 7.76 (broad s, 1H); 7.84 (m, 1H); 8.45 (d, J = 8.3 Hz, 1H); Mass spectrum: ES: m/z = 636 [M + H]⁺; m/z = 634 ([M − H]⁻; base peak); m/z = 680 [M + HCO₂H − H]⁻; Elemental analysis: Calculated: C: 54.71%- H: 4.91%- N: 6.60%- S: 10.07%; Measured: C: 54.76%- H: 4.92%- N: 6.62%- S: 9.73% |
| 12 | 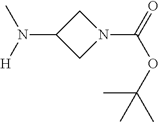 | H | ¹H NMR spectrum (400 MHz; (□ in ppm); (DMSO- d6); referenced at 2.50 ppm): 1.41 (s, 9H); 2.72 (t, J = 7 Hz, 2H); 2.98 (s, 3H); 3.35 (m, 2H); 3.88 (m, 2H); 4.13 (t, J = 8 Hz, 2H); 4.38 (s, 1H); 4.66 (m, 1H); 4.75 (m, 1H); 7.34 (m, 8H); 7.53 (m, 2H); 7.81 (s, 1H); 7.87 (m, 1H); 9.01 (d, J = 7 Hz, 1H); Mass spectrum: ES m/z = 659 (MH⁺) |
| 13 | 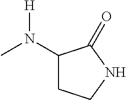 | H | ¹H NMR spectrum (400 MHz; (□ in ppm); (DMSO- d6); referenced at 2.50 ppm): 2.02 (m, 1H); 2.35 (m, 1H); 2.71 (m, 2H); 2.97 (s, 3H); 3.21-3.38 (m, 4H); 4.37 (s, 1H); 4.54 (m, 1H); 4.73 (m, 1H); 7.26- 7.38 (m, 8H); 7.44-7.56 (m, 2H); 7.79 (broad s, 1H); 7.84 (broad s, 1H); 7.85 (m, 1H); 8.75 (d, J = 8.3 Hz, 1H); Mass spectrum: ES: m/z = 587 [M + H]⁺; m/z = 585 [M − H]⁻; m/z = 631 ([M + HCO₂H − H]⁻, base peak); Elemental analysis: Calculated: C: 57.24%- H: 4.80%- N: 9.54%- S: 5.46%; Measured: C: 56.87%- H: 4.94%- N: 9.04%- S: 5.18- H₂O: 0.48% |
| 14 | 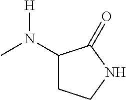 | F | ¹H NMR spectrum (400 MHz; (□ in ppm); (DMSO- d6); referenced at 2.50 ppm): 2.01 (m, 1H); 2.36 (m, 1H); 2.74 (m, 2H); 3.00 (s, 3H); 3.24 (m, 2H); 3.36 (m, 2H); 4.40 (s, 1H); 4.54 (m, 1H); 4.72 (m, 1H); 7.30-7.38 (m, 8H); 7.44 (dt, J = 9.3; 2.1 Hz, 1H); 7.67 (broad s, 1H); 7.68 (m, 1H); 7.86 (s, 1H); 8.83 (d, J = 8.3 Hz, 1H); Mass spectrum: ES: m/z = 605 [M + H]⁺; m/z = 603 [M − H]⁻; m/z = 649 [M + HCO₂H − H]⁻; Elemental analysis: Calculated: C: 55.54%- H: 4.49%- N: 9.25%- S: 5.30%; Measured: C: 55.77%- H: 4.72%- N: 8.91%- S: 4.93% |
| 15 | 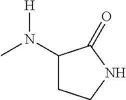 | F | Chiral (+); ¹H NMR spectrum (400 MHz; (□ in ppm); (DMSO- d6); referenced at 2.50 ppm): 2.00 (m, 1H); 2.35 (m, 1H); 2.74 (m, 2H); 3.00 (s, 3H); 3.25 (m, 2H); 3.36 (m, 2H); 4.40 (s, 1H); 4.53 (m, 1H); 4.72 (m, 1H); 7.31 (d, J = 8.6 Hz, 4H); 7.36 (d, J = 8.6 Hz, 4H); 7.44 (broad d, J = 9.5 Hz, 1H); 7.64-7.74 (m, 2H); 7.87 (broad d, 1H); 8.84 (broad d, J = 8.6 Hz, 1H); Mass spectrum: ES: m/z = 605 [M + H]⁺; m/z = 603 [M − H]⁻; Elemental analysis: Calculated: C: 55.54%- H: 4.49%- N: 9.25%- S: 5.30%; Measured: C: 55.32%- H: 4.86%- N: 8.92%- S: 5.06%; Optical rotation: □_{D} = +7.4 +/− 0.5 (c = 0.482, DMSO) |
| 16 | 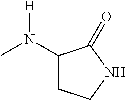 | F | Chiral (−); ¹H NMR spectrum (400 MHz; (□ in ppm); (DMSO- d6); referenced at 2.50 ppm): 2.00 (m, 1H); 2.34 (m, 1H); 2.74 (m, 2H); 3.00 (s, 3H); 3.25 (m, 2H); 3.36 (m, 2H); 4.40 (s, 1H); 4.53 (m, 1H); 4.72 (m, 1H); 7.31 (d, J = 8.6 Hz, 4H); 7.35 (d, J = 8.6 Hz, 4H); 7.44 (broad d, J = 9.3 Hz, 1H); 7.62-7.72 (m, 2H); 7.87 (broad s, 1H); 8.84 (d, J = 8.3 Hz, 1H); Mass spectrum: ES: m/z = 605 [M + H]⁺; m/z = 603 [M − H]⁻; m/z = 649 [M + HCO₂H − H]⁻; Elemental analysis: Calculated: C: 55.54%- H: 4.49%- N: 9.25%- S: 5.30%; Measured: C: 55.21%- H: 4.73%- N: 9.07%- S: 4.95%; Optical rotation: □_{D} = −9.4 +/− 0.6 (c = 0.433, DMSO) |

The compounds according to the invention have been the subject of pharmacological assays which make it possible to determine the activity with respect to human CB1-type cannabinoid receptors. The effectiveness of the compounds of formula (I) was determined in a functional test in which the activity of the CB1 cannabinoid receptors is measured (intracellular cyclic AMP test). The test for detecting intracellular cyclic AMP in U373MG cells naturally expressing the human CB1 receptor was carried out as described in the reference: Bouaboula et al., 1995, J. Biol. Chem. 270:13973-13980. The HTRF cAMP Dynamic Kit from CisBio was used to quantify the intracellular cyclic AMP. In this test, the IC₅₀ values are between 0.001 μM and 1 μM.

For example, Compounds Nos. 9, 14, 16 and 2 showed IC₅₀ values of 130, 9, 7 and 47 nM, respectively.

Other assays consisting in measuring the in vivo activity of the compounds of the invention were carried out. Their antagonist activity was shown by means of the model of hypothermia induced by a CB1 cannabinoid receptor agonist (racemic CP55,940 (1RS, 3RS, 4RS)-3-[hydroxy-2-(1,1-dimethylheptyl)phenyl]-4-(3-hydroxypropyl)cyclohexan-1-ol) at a dose of 1.25 mg/kg) in mice, according to the method described by Pertwee R. G. in Marijuana 84, Harvey D. J. eds. Oxford IRL Press, 263-277 (1985).

Their antagonist activity was also shown by means of the model of inhibition of gastrointestinal transit induced by racemic CP55,940 (1RS, 3RS, 4RS)-3-[hydroxy-2-(1,1-dimethylheptyl)phenyl]-4-(3-hydroxypropyl)cyclohexan-1-ol) in mice, according to the method described by Rinaldi-Carmona et al., J. Pharmacol. Exp. Ther. 2004, 310, 905-914. Briefly, male CD1 mice receive the test product per os 30 minutes or 2 hours before administration of the racemic CP55,940 agonist ((1RS, 3RS, 4RS)-3-[hydroxy-2-(1,1-dimethylheptyl)phenyl]-4-(3-hydroxypropyl)cyclohexan-1-ol) (0.15 mg/kg ip in 10% cremophor). After a further 30 minutes, the animals receive a charcoal bolus po. Thirty minutes later, the animals are sacrificed by euthanasia (CO₂/O₂) and the intestine is dissected. The progression of the charcoal bolus in the intestine is expressed as percentage of the total length of the intestine.

For example, Compounds Nos. 2 at 2 mg/kg and 5, at 1 mg/kg, showed a percentage inhibition of 57% and 39%, respectively, at 3 hours after administration of the product. Consequently, the compounds of the invention of formula (I) are in vitro and in vivo CB1-type cannabinoid receptor antagonists. Some compounds are active in vivo both with regard to the hypothermia test and to transit test, and some compounds show activities split between the hypothermia test and the transit test.

Thus, the compounds of the invention can be used in the treatment or prevention of diseases involving CB1 cannabinoid receptors. These compounds exhibit a peripheral activity dissociated from the central activity.

For example, and without implying limitation, the compounds of formula (I) are of use as psychotropic medicaments, in particular for the treatment of psychiatric disorders, including anxiety, depression, mood disorders, insomnia, delirium disorders, obsessive disorders, psychoses in general, schizophrenia, attention deficient hyperactivity disorders (ADHD) in hyperkinetic children (MBD), and for the treatment of disorders related to the use of psychotropic substances, in particular in the case of substance abuse and/or substance dependence, including alcohol dependence and nicotine dependence, and withdrawal disorders. The compounds of formula (I) according to the invention can be used as medicaments for the treatment of migraine, stress, illnesses of psychosomatic origin, panic attacks, epilepsy, movement disorders, in particular dyskinesias or Parkinson's disease, shaking and dystonia.

The compounds of formula (I) according to the invention can be used as medicaments for skin cancer and for protecting the skin.

The compounds of formula (I) according to the invention can also be used as medicaments in the treatment of memory disorders, cognitive disorders, in particular in the treatment of cognitive disorders related to senile dementia, to Alzheimer's disease, to schizophrenia and to neurodegenerative diseases, and also in the treatment of attention or vigilance disorders.

Furthermore, the compounds of formula (I) may be of use as neuroprotective agents, in the treatment of ischaemia, and of cranial trauma and the treatment of neurodegenerative diseases: including Huntington's chorea or Tourrette's syndrome.

The compounds of formula (I) according to the invention can be used as medicaments in the treatment of pain: neuropathic pain, acute peripheral pain, chronic pain and pain of inflammatory origin.

The compounds of formula (I) according to the invention can be used as medicaments in the treatment of appetite disorders, appetency disorders (craving for sugars, carbohydrates, drugs, alcohols or any appetizing substance) and/or eating disorders, in particular for the treatment of bulimia and also for the treatment of type II diabetes or non-insulin-dependent diabetes and for the treatment of dyslipidamia and metabolic syndrome. Thus, the compounds of formula (I) according to the invention are of use in the treatment of obesity and the risks associated with obesity, in particular cardiovascular risks.

Furthermore, the compounds of formula (I) according to the invention can be used as medicaments in the treatment of gastrointestinal disorders, diarrhea disorders, ulcers, vomiting, bladder and urinary disorders, disorders of endocrine origin, cardiovascular disorders, hypotension, haemorrhagic shock, septic shock, cirrhosis, hepatic fibrosis, steatohepatitis and hepatic steatosis, irrespective of the aetiology of these conditions: in particular, virus, alcohol, medicament, chemical product, autoimmune disease, obesity, diabetes, congenital metabolic disease (haemochromatosis, alpha-1 antitrypsin deficiency. Wilson's disease, etc.), chronic liver cirrhosis, fibrosis, non-alcoholic steatohepatitis (NASH), asthma, chronic obstructive pulmonary diseases. Raynaud's syndrome, glaucoma, fertility disorders, inflammatory phenomena, inflammatory diseases, immune system diseases, in articular autoimmune or neuroinflammatory diseases such as rheumatoid arthritis, reactive arthritis, diseases which result in demyelination, multiple sclerosis, infectious and viral diseases such as encephalitis, cerebral strokes, and also as medicaments for anticancer chemotherapy, for the treatment of Guillain-Barre syndrome, and for the treatment of osteoporosis and sleep apnea.

According to one of its aspects, the present invention relates to the use of a compound of formula (I), of the pharmaceutically acceptable salts thereof, and of the solvates and hydrates thereof, for the treatment of the disorders and diseases indicated above.

According to another of its aspects, the present invention relates to pharmaceutical compositions comprising, as active ingredient, a compound according to the invention. These pharmaceutical compositions contain an effective dose of at least one compound according to the invention, or a pharmaceutically acceptable salt of said compound, and also at least one pharmaceutically acceptable excipient.

Said excipients are chosen, according to the pharmaceutical form and the method of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or the salt thereof, can be administered in unit administration form, as a mixture with conventional pharmaceutical excipients for the treatment of the disorders or diseases mentioned above.

Suitable unit administration forms comprise oral forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical application, the compounds according to the invention may be used in creams, gels, ointments or lotions.

By way of example, a unit administration form of a compound according to the invention in tablet form may comprise the following components:

| | | |
|---|---|---|
| Compound according to the invention | 50.0 | mg |
| Mannitol | 223.75 | mg |
| Sodium croscarmellose | 6.0 | mg |
| Maize starch | 15.0 | mg |
| Hydroxypropylmethylcellulose | 2.25 | mg |
| Magnesium stearate | 3.0 | mg |

There may be specific cases where higher or lower doses are appropriate; such dosages do not depart from the context of the invention. According to customary practice, the dosage suitable for each patient is administered by the physician according to the method of administration and the weight and response of said patient.

According to another of its aspect, the present invention also relates to a method for treating the pathologies indicated above, which comprises the administration, to a patient, of an effective dose of a compound according to the invention or of a pharmaceutically acceptable salt thereof.

1. A compound of formula (I) 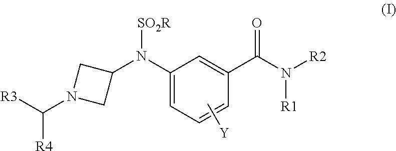
in which:
R represents a (C₁-C₆)alkyl group or a halo(C₁-C₆)alkyl group;
R1 represents a hydrogen atom;
R2 represents a heterocycle group linked via a carbon atom, or a heterocycle-(C₁-C₄)alkyl group, these groups being optionally substituted with one or more atoms or groups chosen from a halogen, a hydroxyl, oxo, cyano, NH₂, C(O)NH₂, a (C₁-C₆)alkyl group, a halo(C₁-C₆)alkyl group, a (C₁-C₆)alkoxy group, a halo(C₁-C₆)alkoxy group or a COO(C₁-C₆)alkyl group;
R3 and R4 represent, independently of one another, a phenyl group optionally substituted with one or more atoms or groups chosen from a halogen, a cyano, a (C₁-C₆)alkyl group, a halo(C₁-C₆)alkyl group, a (C₁-C₆)alkoxy group or a halo(C₁-C₆)alkoxy group;
Y represents a hydrogen atom, a halogen, a cyano, a (C₁-C₆)alkyl group, a halo(C₁-C₆)alkyl group, a (C₁-C₆)alkoxy group, a halo(C₁-C₆)alkoxy group or a (C₁-C₆)alkylS(O)ₚ group;
p is between 0 and 2;
in the form of a base or of an addition salt with an acid.

2. The compound according to claim 1, wherein:
R represents a methyl,
R3 and R4 each represent a phenyl group substituted with a chlorine atom in the para-position;
Y represents a hydrogen atom or a halogen:
R1 represents a hydrogen atom;
R2 represents a heterocycle group linked via a carbon atom or a heterocycle-(C₁-C₄)alkyl group and the heterocycle represents a tetrahydrothiophene, piperidine, tetrahydrothiopyran, azetidine, pyrrolidine or imidazolidine, which are optionally substituted with one or more (C₁-C₆)alkyl, COO(C₁-C₆)alkyl or oxo groups;
in the form of a base or of an addition salt with an acid.

3. The compound according to claim 1, chosen from:
3-[{1-[bis(4-chlorophenyl)methyl]azetidin-3-yl}(methyl sulphonyl)amino]-N-[3-(2-oxopyrrolidin-1-yl)propyl]benzamide
3-[{1-[bis(4-chlorophenyl)methyl]azetidin-3-yl}(methylsulphonyl)amino]-N-(1,1-dioxidotetrahydrothiophen-3-yl)benzamide
3-[{1-[bis(4-chlorophenyl)methyl]azetidin-3-yl}(methylsulphonyl)amino]-N-[(1-ethylpyrrolidin-2-yl)methyl]benzamide hydrochloride (1:2)
tert-butyl 4-[({3-[{1-[bis(4-chlorophenyl)methyl]azetidin-3-yl}(methyl sulphonyl)amino]phenyl}carbonyl)amino]piperidine-1-carboxylate
(−)-3-[{1-[bis(4-chlorophenyl)methyl]azetidin-3-yl}(methylsulphonyl)amino]-N-{[1-ethyl-pyrrolidin-2-yl]methyl}benzamide
(+)-3-[{1-[bis(4-chlorophenyl)methyl]azetidin-3-yl}(methylsulphonyl)amino]-N-{[1-ethyl-pyrrolidin-2-yl]methyl}benzamide
(−)-3-[{1-[bis(4-chlorophenyl)methyl]azetidin-3-yl}(methylsulphonyl)amino]-N-[1,1-dioxidotetrahydrothiophen-3-yl]benzamide
(+)-3-[{1-[bis(4-chlorophenyl)methyl]azetidin-3-yl}(methylsulphonyl)amino]-N-[1,1-dioxidotetrahydrothiophen-3-yl]benzamide
3-[{1-[bis(4-chlorophenyl)methyl]azetidin-3-yl}(methylsulphonyl)amino]-N-[2-(2-oxoimidazolidin-1-yl)ethyl]benzamide
3-[{1-[bis(4-chlorophenyl)methyl]azetidin-3-yl}(methylsulphonyl)amino]-N-(tetrahydro-2H-thiopyran-4-yl)benzamide
3-[{1-[bis(4-chlorophenyl)methyl]azetidin-3-yl}(methylsulphonyl)amino]-N-(1,1-dioxidotetrahydro-2H-thiopyran-4-yl)benzamide
tert-butyl 3-[({3-[{1-[bis(4-chlorophenyl)methyl]azetidin-3-yl}(methyl-sulphonyl)amino]phenyl}carbonyl)amino]azetidine-1-carboxylate
3-[{1-[bis(4-chlorophenyl)methyl]azetidin-3-yl}(methylsulphonyl)amino]-N-(2-oxopyrrolidin-3-yl)benzamide
3-[{1-[bis(4-chlorophenyl)methyl]azetidin-3-yl}(methylsulphonyl)amino]-5-fluoro-N-(2-oxopyrrolidin-3-yl)benzamide
(+)-3-[{1-[bis(4-chlorophenyl)methyl]azetidin-3-yl}(methylsulphonyl)amino]-5-fluoro-N-[2-oxopyrrolidin-3-yl]benzamide
(−)-3-[{1-[bis(4-chlorophenyl)methyl]azetidin-3-yl}(methylsulphonyl)amino]-5-fluoro-N-[2-oxopyrrolidin-3-yl]benzamide.

4. (canceled)

5. A pharmaceutical composition, comprising the compound of claim 1.

6. A method of treating or preventing psychiatric disorders, substance dependence and withdrawal, tobacco withdrawal, cognitive and attention disorders, and acute and chronic neurodegenerative diseases in a patient in need thereof comprising administering to said patient a therapeutically effective amount of the pharmaceutical composition of claim 5.

7. A method of treating or preventing metabolic disorders, appetency disorders, appetite disorders, obesity, diabetes, metabolic syndrome, dyslipidemia and sleep apnea in a patient in need thereof comprising administering to said patient a therapeutically effective amount of the pharmaceutical composition of claim 5.

8. A method for treating or preventing pain, neuropathic pain and neuropathic pain induced by anticancer drugs in a patient in need thereof comprising administering to said patient a therapeutically effective amount of the pharmaceutical composition of claim 5.

9. A method for treating or preventing gastrointestinal disorders, vomiting, ulcers, diarrhea disorders, bladder and urinary disorders, disorders of endocrine origin, cardiovascular disorders, hypotension, haemorrhagic shock, septic shock, liver diseases, chronic liver cirrhosis, fibrosis, non-alcoholic steatohepatitis (NASH), steatohepatitis and hepatic steatosis, irrespective of the aetiology of these conditions (alcohol, medicament, chemical product, autoimmune disease, obesity, diabetes, congenital metabolic disease) in a patient in need thereof comprising administering to said patient a therapeutically effective amount of the pharmaceutical composition of claim 5.

10. A method for treating or preventing immune system diseases, rheumatoid arthritis, demyelination, multiple sclerosis, inflammatory diseases in a patient in need thereof comprising administering to said patient a therapeutically effective amount of the pharmaceutical composition of claim 5.

11. A method for treating or preventing Alzheimer's disease, Parkinson's disease, schizophrenia, and cognitive disorders associated with schizophrenia, with diabetes, with obesity or with metabolic syndrome in a patient in need thereof comprising administering to said patient a therapeutically effective amount of the pharmaceutical composition of claim 5.

12. A method for treating or preventing asthma, chronic obstructive pulmonary diseases, Raynaud's syndrome, glaucoma and fertility disorders in a patient in need thereof comprising administering to said patient a therapeutically effective amount of the pharmaceutical composition of claim 5.

13. A method for treating or preventing infectious and viral diseases, such as encephalitis, cerebral strokes, Guillain-Barré syndrome, osteoporosis and sleep apnea, and for anticancer therapy in a patient in need thereof comprising administering to said patient a therapeutically effective amount of the pharmaceutical composition of claim 5.

14. A process for preparing the compound according to claim 1 comprising reacting an acid derivative of Formula 5 with an amine derivative of Formula 6 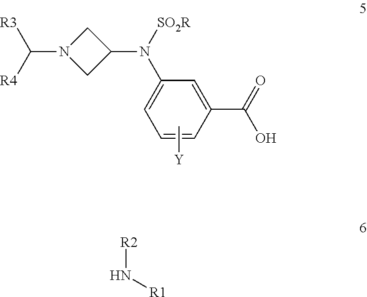
in an inert solvent, in the presence of a coupling agent and, optionally, of an additive which prevents racemization, optionally deprotecting the product, and isolating and optionally converting said product to an addition salt with an acid.
